(19) United States
(12) Patent Application Publication
(10) Pub. No.: US 2023/0001266 A1
(43) Pub. Date: Jan. 5, 2023

(54) EXERCISE MENU EVALUATION APPARATUS, METHOD, AND COMPUTER READABLE MEDIUM

(71) Applicant: NEC Corporation, Minato-ku, Tokyo (JP)

(72) Inventor: Tomohiro MIWA, Tokyo (JP)

(73) Assignee: NEC Corporation, Minato-ku, Tokyo (JP)

(21) Appl. No.: 17/784,725

(22) PCT Filed: Dec. 10, 2020
(86) PCT No.: PCT/JP2020/046143

§ 371 Date: Jun. 13, 2022

(30) Foreign Application Priority Data
Dec. 26, 2019 (JP) 2019-236956

(51) Int. Cl. A63B 24/00; A63B 71/06

(52) U.S. Cl. CPC A63B 24/0062; A63B 24/0006; A63B 71/0622; A63B 2024/0068; A63B 2024/0071; A63B 2220/05; A63B 2220/62; A63B 2220/806

(57) ABSTRACT

Evaluation value calculation unit calculates, based on time-series changes in exercise information on each of a plurality of users who have exercised in accordance with an exercise menu, an evaluation value of the exercise menu. Evaluation result generation unit compares an evaluation value of an exercise menu with a reference value. The evaluation result generation unit generates results of evaluating the exercise menu based on the results of comparing the evaluation value with the reference value.

### TECHNICAL FIELD

The present disclosure relates to an exercise menu evaluation apparatus, a method, and a computer readable medium.

### BACKGROUND ART

In the field of medicine, nursing care, or health care, it is quite common for professionals such as physical therapists or trainers to create exercise programs and provide them for users for the purpose of improving and strengthening physical functions, and preventive care. Exercise programs include a combination of one or more exercise menus. Each exercise menu includes one type of exercise motion.

Many systems for creating appropriate exercise programs depending on users and providing them to the respective users have been proposed in related art. For example, Patent Literature 1 discloses an exercise program presenting system. In the exercise program presenting system, a server accumulates personal information of users who have performed exercise programs, the period during which the exercise programs have been performed, and the results of measuring effects of the exercise programs. The exercise program presenting system extracts, when creating an exercise program for a new user, previous cases in which the personal information on the users in these cases is similar to the personal information on the new user and automatically creates an exercise program that matches the features of this user.

### CITATION LIST

### Patent Literature

[Patent Literature 1] Japanese Unexamined Patent Application Publication No. 2017-27192
[Patent Literature 2] Japanese Unexamined Patent Application Publication No. 2018-198707

### SUMMARY OF INVENTION

### Technical Problem

In Patent Literature 1, when the quality of an exercise menu itself provided for a user is low, the quality of the exercise program including this exercise menu naturally becomes low. In order to provide exercise programs each having a higher quality, professionals need to appropriately update exercise menus to be used for creating the exercise programs to improve the quality of the exercise menu group. However, if the number of exercise menus used in the exercise program is large, it is difficult to determine which exercise menus in the large number of exercise menus are low-quality menus. Further, in Patent Literature 1, the quality of each of the exercise menus is not taken into account, and thus an exercise program including low-quality exercise menus may be presented.

As another related art, Patent Literature 2 discloses a teaching compatibility determining device. Patent Literature 2 discloses a program for recording information on exercises performed by exercisers who participate in sports such as swimming and track and field. A computer disclosed in Patent Literature 2 acquires measurement values by measuring exercises performed by an exerciser and acquires exercise information regarding the exercises performed by the exerciser based on the measurement values. The computer displays the acquired exercise information in association with a target value of exercises performed by the exerciser.

Patent Literature 2 displays exercise information on an exerciser in association with the target value in order to let a user know which part of the body of the exerciser should be improved by an exercise and how much the part of the body should be improved by the exercise. In Patent Literature 2, however, the effectiveness of the exercise itself is not evaluated. Therefore, regarding Patent Literature 2, like Patent Literature 1, it is difficult to determine which exercise menus in the large number of exercise menus are low-quality menus.

In view of the aforementioned circumstances, the aim of the present disclosure is to provide an exercise menu evaluation apparatus, a method, and a computer readable medium capable of evaluating effectiveness of exercise menus.

### Solution to Problem

In order to attain the above object, the present disclosure provides an exercise menu evaluation apparatus as a first aspect. The exercise menu evaluation apparatus includes: evaluation value calculation means for calculating, based on time-series changes in exercise information on each of a plurality of users who have exercised in accordance with an exercise menu, an evaluation value of the exercise menu; and evaluation result generation means for comparing the evaluation value of the exercise menu with a reference value and generating results of evaluating the exercise menu based on the results of the comparison.

The present disclosure provides an exercise menu evaluation method as a second aspect. The exercise menu evaluation method includes: calculating, based on time-series changes in exercise information on each of a plurality of users who have exercised in accordance with an exercise menu, an evaluation value of the exercise menu; and comparing the evaluation value of the exercise menu with a reference value and generating results of evaluating the exercise menu based on the results of the comparison.

The present disclosure provides a computer readable medium according to a third aspect. The computer readable medium stores a program for causing a computer to execute the processing of: calculating, based on time-series changes in exercise information on each of a plurality of users who have exercised in accordance with an exercise menu, an evaluation value of the exercise menu; and comparing the evaluation value of the exercise menu with a reference value and generating results of evaluating the exercise menu based on the results of the comparison.

### Advantageous Effects of Invention

An exercise menu evaluation apparatus, a method, and a computer readable medium according to the present disclosure are able to evaluate effectiveness of exercise menus.

### BRIEF DESCRIPTION OF DRAWINGS

FIG. 1 is a block diagram showing a schematic configuration of an exercise menu evaluation apparatus according to the present disclosure;

FIG. 2 is a block diagram showing an exercise program presenting system including the exercise menu evaluation apparatus according to one example embodiment of the present disclosure;

FIG. 3 is a diagram showing a specific example of exercise menu information;

FIG. 4 is a diagram showing a specific example of user information;

FIG. 5 is a diagram showing a specific example of an exercise program provided for a user;

FIG. 6 is a diagram showing a specific example of exercise data;

FIG. 7 is a block diagram showing a configuration example of the exercise menu evaluation apparatus;

FIG. 8 is a graph showing a state of change in a maximum range of motion of a user n who has performed an exercise menu k;

FIG. 9 is a diagram showing one example of acquisition of a maximum range of motion at an intermediate time point;

FIG. 10 is a flowchart showing a procedure of operations in the exercise menu evaluation apparatus;

FIG. 11 is a block diagram showing a configuration example of an exercise menu evaluation apparatus according to a modified example;

FIG. 12 is a diagram showing a specific example of data stored in an exercise record in the modified example; and

FIG. 13 is a block diagram showing a configuration example of a computer apparatus.

### DESCRIPTION OF EMBODIMENTS

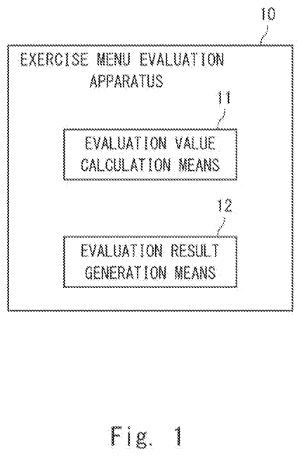

Prior to giving the description of an example embodiment of the present disclosure, an outline of the present disclosure will be described. FIG. 1 schematically shows a configuration of an exercise menu evaluation apparatus according to the present disclosure. An exercise menu evaluation apparatus 10 includes evaluation value calculation means 11 and evaluation result generation means 12.

The evaluation value calculation means calculates an evaluation value of an exercise menu based on time-series changes in exercise information on each of a plurality of users who have exercised in accordance with the exercise menu. The evaluation result generation means 12 compares the evaluation value of the exercise menu with a reference value. The evaluation result generation means 12 generates results of evaluating the exercise menu based on the results of the comparison.

In the present disclosure, the evaluation value calculation means 11 calculates, based on exercise information on each of a plurality of users who have performed exercise in one exercise menu, an evaluation value of the exercise menu. The evaluation result generation means 12 generates results of the evaluation based on results of comparing the calculated evaluation value with the reference value. According to this procedure, it is possible to evaluate effectiveness of the exercise menu from the record of the exercise in the exercise menu that the users have performed.

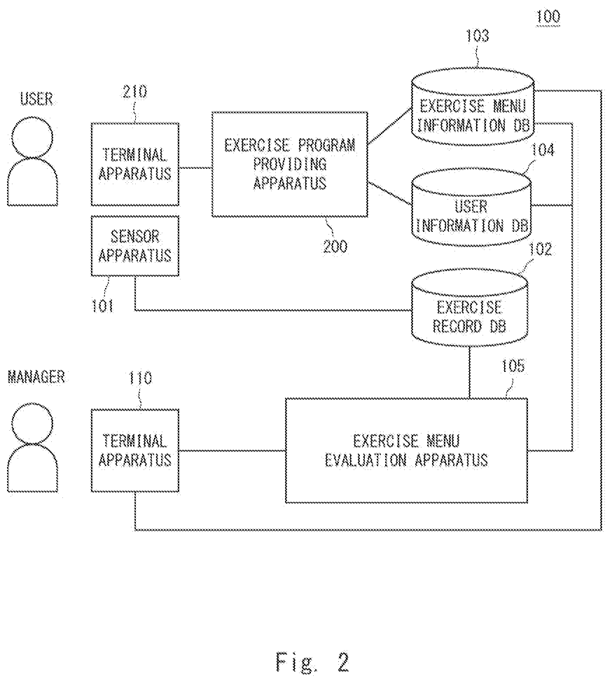

Hereinafter, with reference to the drawings, the example embodiment of the present disclosure will be described. FIG. 2 shows an exercise program presenting system including the exercise menu evaluation apparatus according to one example embodiment of the present disclosure. An exercise program presenting system 100 includes a sensor apparatus 101, an exercise record database (DB) 102, an exercise menu information DB 103, a user information DB 104, an exercise menu evaluation apparatus 105, and an exercise program providing apparatus 200.

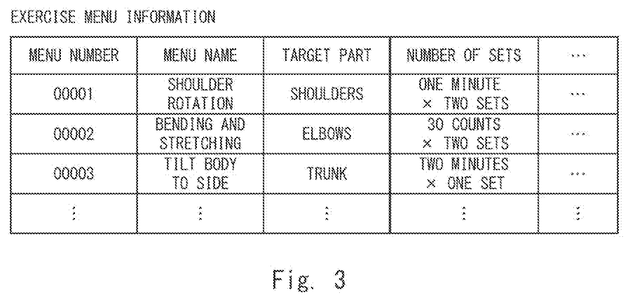

The exercise menu information DB 103 stores information regarding exercise menus used in an exercise program (exercise menu information). FIG. 3 shows a specific example of the exercise menu information stored in the exercise menu information DB 103. In this example, the exercise menu information includes a menu number, a target part, and the number of sets. The menu number indicates identification information of an exercise menu. The target part indicates the part of the body intended to be trained in the exercise menu. For example, the exercise menu identified by the menu number “00001” is “shoulder rotation”, which is an exercise menu in which “shoulders” are the target part. “The number of sets” indicates the number of times of repetition of exercise, an exercise duration time or the like. For example, the exercise menu identified by the menu number “00001” is an exercise menu in which two sets of exercise, each including one-minute exercise, is performed.

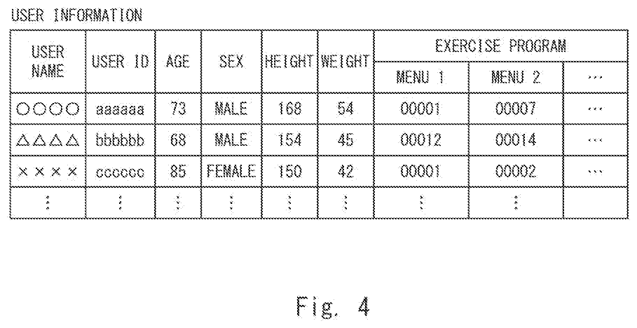

The user information DB 104 stores information on each user (user information) who performs an exercise menu in accordance with an exercise program. FIG. 4 shows a specific example of user information stored in the user information DB 104. In this example, the user information includes the user name, the user identifier (ID), age, sex, height, and weight of each user. Further, the user information includes an exercise program created for each user. The exercise program includes one or more exercise menus selected from a plurality of exercise menus stored in the exercise menu information DB 103. In the example shown in FIG. 4, the user information DB 104 stores, for example, an exercise program including exercise menus whose menu numbers are “00001” and “00007” for the user whose user ID is “aaaaa”.

The exercise program providing apparatus 200 generates exercise programs. The exercise program providing apparatus 200 generates exercise programs provided for the respective users based on, for example, information on each user such as age, sex, height, and weight stored in the user information DB 104. The exercise menu information DB 103 stores, for example, information regarding M (M is an integer of 1 or more) kinds of exercise menus. The exercise program providing apparatus 200 generates the exercise program by combining one or more exercise menus selected from M types of exercise menus. The exercise program providing apparatus 200 is formed, for example, as a computer apparatus including a processor and a memory. The functions of the exercise program providing apparatus 200 may be implemented, for example, by a processor operating in accordance with a program loaded from the memory.

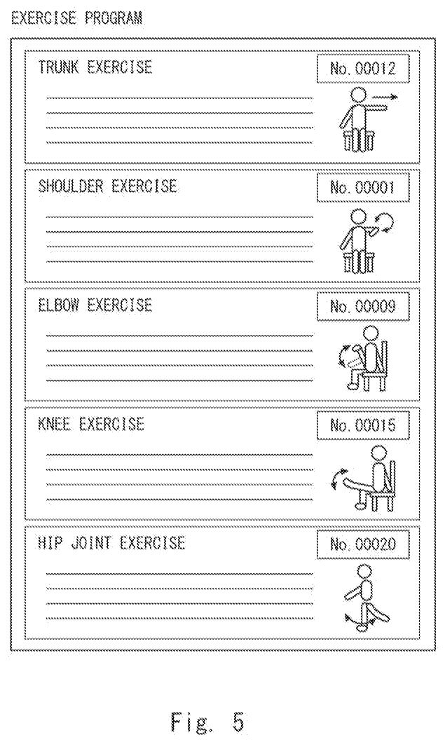

FIG. 5 shows a specific example of the exercise program provided for a user. In this example, the exercise program includes five exercise menus whose target parts are different from one another. Specifically, the exercise program includes five exercise menus whose target parts are trunk, shoulders, elbows, knees, and hip joint. The exercise program providing apparatus 200 transmits the exercise program to a terminal apparatus 210 used by the user. The user refers to his/her exercise program displayed on the terminal apparatus 210 and executes the exercise of each exercise menu included in the exercise program. The exercise menus may be provided using, for example, a print medium, video images or the like. The method of providing the exercise menus is not particularly limited.

While exercise menus are assigned to five respective target parts in the example shown in FIG. 5, this example embodiment is not limited thereto. For example, one exercise program may include a plurality of exercise menus whose target part is the same. It is assumed, in this case, that one exercise program does not include a plurality of exercise menus that are exactly the same. The upper limit of the number of exercise menus included in one exercise program may be the same as the number of exercise menus stored in the exercise menu information DB 103.

The sensor apparatus 101 acquires exercise data when the user has performed exercise of each exercise menu in accordance with the exercise program. The sensor apparatus 101 includes, for example, a camera. The camera captures images of the user during the exercise. The sensor apparatus 101 acquires video images of the user who performs exercise as the exercise data. The sensor apparatus 101 may include other sensors such as a depth sensor, an infrared sensor, an acceleration sensor, and a pressure sensor. The sensor apparatus 101 acquires information measured by various sensors as the exercise data. The number of sensors and the types of the sensors used to acquire the exercise data are not particularly limited. The sensor apparatus 101 may include, for example, a plurality of sensors.

The exercise record DB 102 records exercise data that the sensor apparatus 101 has acquired when the user has performed each exercise menu in accordance with the exercise program. The exercise record DB 102 records, for example, for each exercise menu, the exercise data of each user that the sensor apparatus 101 has acquired. The exercise record DB 102 may store, when the sensor apparatus 101 includes a plurality of types of sensors, the exercise data that each sensor acquires.

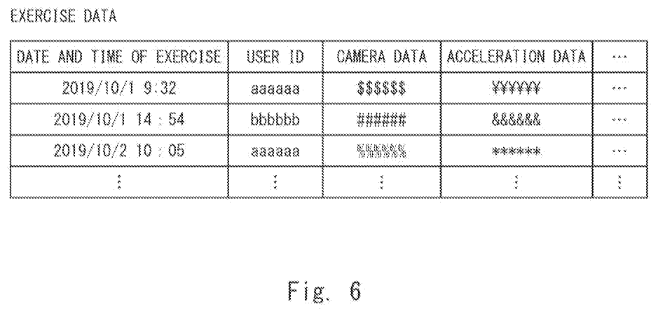

FIG. 6 shows a specific example of exercise data recorded in the exercise record DB 102. The exercise data shown in FIG. 6 is exercise data regarding one exercise menu. In this example, the exercise data includes date and time of exercise, user ID, camera data, and acceleration data. The date and time of exercise indicates the date and time when exercise has been performed. The user ID indicates identification information of the user who has performed exercise of the exercise menu.

The camera data and the acceleration data are data obtained using the sensor apparatus 101. For example, a camera, which is the sensor apparatus 101, transmits video images captured by the camera to the exercise record DB 102 after the user completes exercise of the exercise menu. The exercise record DB 102 stores video data in association with the user ID. A plurality of users continuously perform exercise programs, whereby the exercise record DB 102 accumulates a large amount of exercise data regarding various exercise menus.

The exercise menu evaluation apparatus 105 evaluates the effectiveness of the exercise menus. The exercise menu evaluation apparatus 105 evaluates each of the exercise menus based on data (information) stored in the exercise record DB 102, the exercise menu information DB 103, and the user information DB 104 after, for example, each user has performed the exercise program for a certain period of time. The exercise menu evaluation apparatus 105 transmits the results of the evaluation to a terminal apparatus 110 used by a manager. The exercise menu evaluation apparatus 105 corresponds to the exercise menu evaluation apparatus 10 shown in FIG. 1.

Regarding the period during which a user performs an exercise program, requirements for calculating additional fee for individual function training performed in, for example, nursing care facilities require the content of the training to be reviewed at least once in three months. It is assumed that the period during which the exercise program is performed be set to three months in accordance therewith.

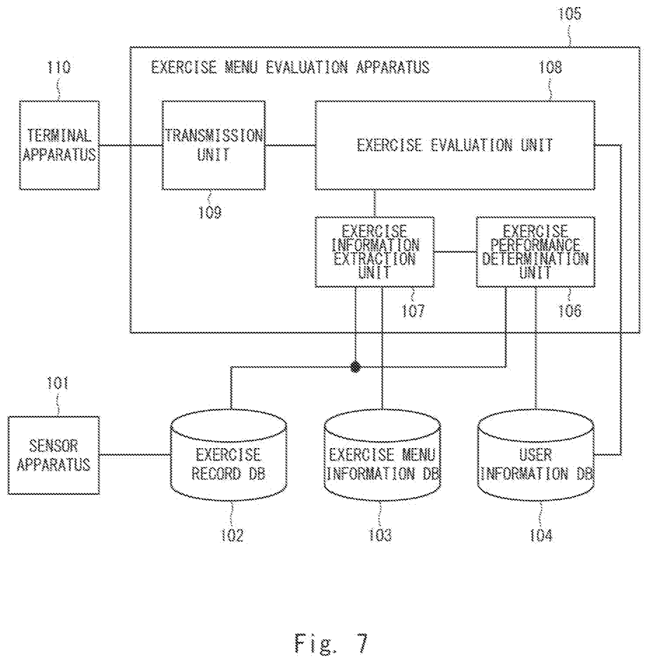

FIG. 7 shows a configuration example of the exercise menu evaluation apparatus 105. The exercise menu evaluation apparatus 105 includes an exercise performance determination unit 106, an exercise information extraction unit 107, an exercise evaluation unit 108, and a transmission unit 109. The exercise menu evaluation apparatus 105 is formed, for example, as a computer apparatus including a processor and a memory. The functions of the respective parts in the exercise menu evaluation apparatus 105 are achieved, for example, by a processor operating in accordance with a program loaded from a memory.

The exercise performance determination unit (exercise performance determination means) 106 refers to the user information DB 104 for each exercise menu stored in the exercise menu information DB 103 and determines whether the exercise program provided for the user includes the exercise menu. In other words, the exercise performance determination unit 106 determines whether or not the user has performed each exercise menu. The exercise performance determination unit 106 notifies the exercise evaluation unit 108 of exercise menus determined to have been performed by the user. The exercise performance determination unit 106 excludes exercise menus determined not to have been performed by the user from the target to be evaluated in the exercise evaluation unit 108.

The exercise performance determination unit 106 sequentially selects, for example, M types of exercise menus one by one. The exercise performance determination unit 106 refers to the user information DB 104 and determines whether or not the selected exercise menus are included in the exercise program provided for the user. The exercise performance determination unit 106 determines, for example, that, among all the M types of exercise menus, K (K≠0) types of exercise menus have been performed. In this case, K types of exercise menus are to be evaluated in the exercise evaluation unit 108. When K=0, no exercise menu is evaluated and the result indicating “no target to be evaluated” is transmitted to the terminal apparatus 110 of the manager.

The exercise information extraction unit (exercise information extraction means) 107 extracts exercise information from exercise data recorded in the exercise record DB 102. The exercise information extraction unit 107 extracts, for example, exercise information from the exercise data for an exercise menu k (K=1, 2, 3, . . . , K) included in the K types of exercise menus to be evaluated, by performing appropriate processing in accordance with the type of exercise data stored in the exercise record DB 102.

Consider a case, for example, in which a camera is used as the sensor apparatus 101. The exercise information extraction unit 107 extracts at least one of “range of motion”, “number of times of exercise (per unit time)”, or “posture” from video images, which are the exercise data, as exercise information. In the following description, an example in which the exercise information extraction unit 107 extracts the range of motion of the target part will be explained. The exercise information extraction unit 107 extracts, for example, skeleton information of the user from the video images, and generates the index indicating the maximum range of motion of the target part of each exercise menu.

The exercise evaluation unit 108 acquires exercise information on each user from the exercise information extraction unit 107. The exercise evaluation unit 108 evaluates, regarding an exercise menu to be evaluated, the exercise menu based on the exercise information on each user. The exercise evaluation unit 108 calculates the evaluation value of the exercise menu based on time-series changes in exercise information on each of a plurality of users who have exercised in accordance with the exercise menu to be evaluated. The exercise evaluation unit 108 compares the evaluation value of the exercise menu with a reference value and generates the results of evaluating the exercise menu based on the results of the comparison. The exercise evaluation unit 108 corresponds to the evaluation value calculation means 11 and the evaluation result generation means 12 shown in FIG. 1.

For example, the exercise evaluation unit 108 selects the exercise menu k from the K types of exercise menus, which are exercise menus to be evaluated. It is assumed, in this example, that the number of users who have performed exercise programs including the exercise menu k is N (N is an integer of 2 or more) and the users have performed the respective exercise programs including the exercise menu k at the same frequency in a certain period of time. The exercise programs performed by the respective users may include the different types of the exercise menus or the different number of the exercise menus, as long as the exercise menu k is included.

The exercise evaluation unit 108 calculates, for each user who has performed the exercise menu k, the evaluation value of the exercise menu k based on values of the maximum range of motion at a plurality of time points in a certain period of time. For example, the exercise evaluation unit 108 may calculate the evaluation value of the exercise menu k based on the value of the maximum range of motion at each of the start time point (first time point) of execution of the exercise menu k, the end time point (second time point) thereof, and an intermediate time point (third time point) between the start time point and the end time point. Note that the time points used for the evaluation are not particularly limited to the aforementioned three time points. The exercise evaluation unit 108 may evaluate the exercise menu k based on the values of the maximum range of motion at a plurality of time points that the manager has freely set.

It is assumed, for example, that the start time point of a certain period of time at which each user has performed the exercise menu k is denoted by tₓ, the intermediate time point is denoted by t_{y}, and the end time point (the latest performance time point) is denoted by t_{z}. The values of the maximum range of motion at the respective time points of one user n (n=1, 2, 3, . . . , N) are denoted by xₙₖ, yₙₖ, and zₙₖ. The exercise evaluation unit 108 calculates the evaluation index regarding the user n based on the amount of change between xₙₖ and yₙₖ and the amount of change between xₙ and zₙₖ and calculates the evaluation value of the exercise menu k based on the evaluation values of the respective users.

If, for example, the period during which the exercise menu k has been performed is six months, the exercise evaluation unit 108 acquires, regarding the user n, the values xₙₖ, yₙₖ, and zₙₖ of the maximum range of motion at the start time point, and the time points after three months and six months since the start of the exercise. The exercise evaluation unit 108 calculates a degree of improvement of the range of motion in a period between the start time point and the intermediate time point by calculating, for example, the difference between the value xₙₖ of the maximum range of motion at the start time point and the value yₙₖ of the maximum range of motion at the intermediate time point. Further, the exercise evaluation unit 108 calculates a degree of improvement of the range of motion in a period between the start time point and the intermediate time point by calculating the difference between the value xₙₖ of the maximum range of motion at the start time point and the value zₙₖ of the maximum range of motion at the end time point. The difference between the maximum ranges of motion is also referred to as an improvement score. The exercise evaluation unit 108 calculates the rate of the improvement score from the start time point to the intermediate time point to the improvement score from the start time point to the end time point. The rate of improvement score indicates the rate of improvement. Specifically, the exercise evaluation unit 108 calculates, regarding the exercise menu k, the rate of improvement pₙₖ of the user n by the following expression.

$${p}_{nk} = \frac{{z}_{nk} - {x}_{nk}}{{y}_{nk} - {x}_{nk}} \qquad [ Expression 1 ]$$

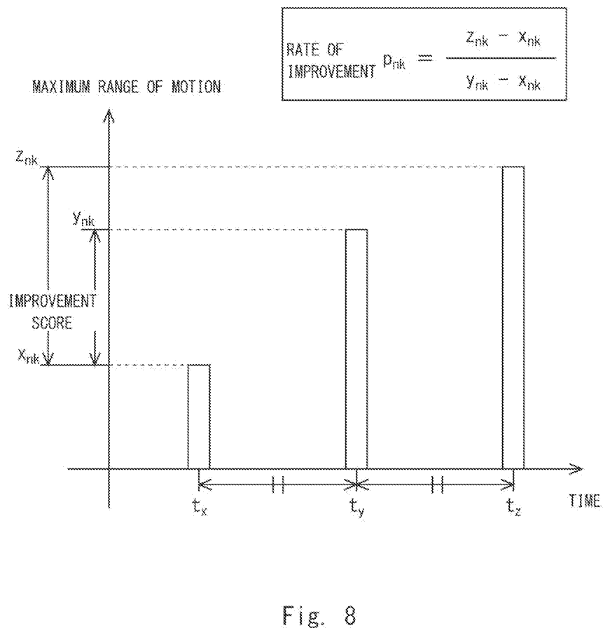

FIG. 8 shows a state of change in the maximum range of motion of the user n who has performed the exercise menu k. In the graph shown in FIG. 8, the horizontal axis indicates a time and the vertical axis indicates the maximum range of motion of the target part of the user n who performs the exercise menu k. The exercise evaluation unit 108 calculates the difference between the maximum range of motion xₙₖ at the start time point tₓ and the maximum range of motion yₙₖ at the intermediate time point t_{y} as the improvement score from the start time point to the intermediate time point. Further, the exercise evaluation unit 108 calculates the difference between the maximum range of motion xₙₖ at the start time point tₓ and the maximum range of motion zₙₖ at the end time point t_{z} as the improvement score from the start time point to the end time point. The exercise evaluation unit 108 calculates the rate of the improvement score as the above rate of improvement pₙₖ.

The above rate of improvement pₙₖ larger than 1 indicates that the maximum range of motion at the end time point is larger than the maximum range of motion at the intermediate time point. The rate of improvement pₙₖ equal to or smaller than 1 indicates that the maximum range of motion has not improved or the maximum range of motion has been decreased when the maximum range of motion at the intermediate time point is compared to that at the end time point. Further, the rate of improvement pₙₖ which is a negative value indicates that the maximum range of motion at the intermediate time point or the end time point is smaller than the maximum range of motion at the start time point.

The exercise record DB 102 stores, for example, the exercise data of the user n at the start time point of a certain period such as six months, and it can be considered that the maximum range of motion xₙₖ can be acquired from this exercise data. However, there may be a case in which the exercise record DB 102 does not store exercise data at time point after three months or six months from the start of exercise. The exercise evaluation unit 108 provides a buffer period, for example, before and after the intermediate time point and the end time point. The exercise evaluation unit 108 may calculate, when the exercise record DB 102 does not store exercise data at the intermediate time point and the end time point, the rate of improvement pₙₖ using the maximum ranges of motion yₙₖ and zₙₖ extracted from the exercise data in the buffer period.

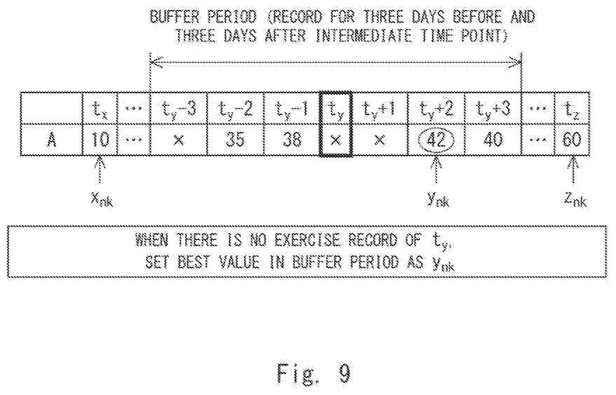

FIG. 9 shows one example of acquisition of the maximum range of motion at the intermediate time point t_{y}. In this example, the exercise record DB 102 does not store exercise data at the intermediate time point t_{z} although it stores exercise data at the start time point tₓ and the end time point t_{z}. The exercise information extraction unit 107 extracts exercise information from exercise data within a buffer period, which includes, for example, three days before the intermediate time point t_{z} and three days after the intermediate time point t_{z}.

In the example shown in FIG. 9, the exercise record DB 102 stores exercise data at each time point of t_{y}−2, t_{y}−1, t_{y}+2, and t_{y}+3. The exercise information extraction unit 107 extracts the maximum range of motion from the exercise data at the above time points. The exercise evaluation unit 108 sets the best value among the maximum ranges of motion in the buffer period of total seven days as the maximum range of motion at the intermediate time point t_{y}. In the example shown in FIG. 9, the value of the maximum range of motion at the time point of t_{y}+2 is the largest. In this case, the exercise evaluation unit 108 calculates the improvement score by setting “42”, which is the value of the maximum range of motion at the time point of t_{y}+2, as yₙₖ.

Regarding the end time point t_{z}, since t_{z} is the latest record, the exercise record DB 102 does not store exercise data at time points later than t_{z}. The exercise evaluation unit 108 may set, for the end time point t_{z}, the best value within a buffer period, which is a period of six days counted back from the end time point t_{z}, as zₙₖ. The exercise evaluation unit 108 does not calculate, if the exercise record DB 102 does not store exercise data of one user in a buffer period, the rate of improvement for this user.

While the exercise evaluation unit 108 sets a buffer period of six days before and after the intermediate time point and before the end time point in the above example, this example embodiment is not limited thereto. The length and the like of the buffer period can be freely set. Further, while the example in which the maximum value of the maximum ranges of motion in the buffer period is determined as the value of the maximum range of motion at the intermediate time point has been described in the above example, this example embodiment is not limited thereto. The exercise evaluation unit 108 may determine the average of the maximum ranges of motion in the buffer period as the value of the maximum range of motion at the intermediate time point or the end time point.

The exercise evaluation unit 108 sets the average of the rates of improvement obtained for all the users who have performed the exercise menu k as the evaluation value pₖ of the exercise menu k. The evaluation value pₖ can be expressed by the following expression using the rate of improvement pₙₖ of the user k.

$${p}_{k} = \frac{1}{N} {\sum }_{n = 1}^{N} {p}_{nk} \qquad [ Expression 2 ]$$

The exercise evaluation unit 108 obtains the above evaluation value pₖ for each of a plurality of exercise menus to be evaluated. The exercise evaluation unit 108 determines, based on the evaluation values of the plurality of exercise menus, the reference value of the evaluation of the effectiveness of the exercise menus. For example, the exercise evaluation unit 108 sets the average of the evaluation values pₖ as the reference value of the exercise menu. The exercise evaluation unit 108 compares the evaluation value of each exercise menu with the reference value and determines the quality (effectiveness) of each exercise menu based on the results of the comparison. The exercise evaluation unit 108 determines, for example, that exercise menus whose evaluation values are smaller than the reference value are low-quality exercise menus.

The exercise evaluation unit 108 generates results of evaluating the exercise menus and outputs the results of the evaluation to the transmission unit 109. The exercise evaluation unit 108 generates, for example, a list of exercise menus determined to have low qualities as the results of the evaluation. Alternatively, the exercise evaluation unit 108 may generate a list in which the exercise menus to be evaluated are rearranged in accordance with the results of the evaluation (evaluation values) as the results of the evaluation. The exercise evaluation unit 108 may output the results of evaluating the exercise menus specified by the manager.

The transmission unit 109 transmits the results of evaluating the exercise menus to the terminal apparatus 110 of the manager. The transmission unit 109 transmits, for example, the list of low-quality exercise menus to the terminal apparatus 110 of the manager. The terminal apparatus 110 displays the results of the evaluation on a display screen. The terminal apparatus 110 displays, for example, exercise menus included in the list of low-quality exercise menus. The manager is able to know which exercise menus have low effectiveness by referring to the results of the evaluation such as the list of low-quality exercise menus. The manager is able to efficiently correct or update exercise menus by reviewing the content of the low-effective exercise menus.

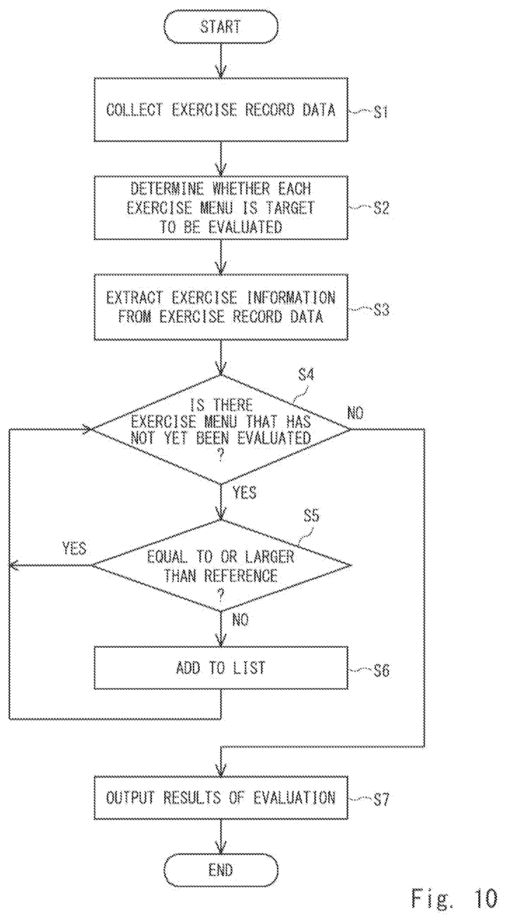

Next, a procedure of operations in the exercise menu evaluation apparatus 105 (exercise menu evaluation method) will be described. FIG. 10 shows a procedure of operations in the exercise menu evaluation apparatus 105. Each of the plurality of users performs exercise in the exercise menus included in the exercise program provided for him or her. The exercise record DB 102 collects exercise data of each user and stores the collected exercise data (Step S1). The exercise record DB 102 collects and stores, for example, a large amount of exercise data.

The exercise performance determination unit 106 determines whether or not each exercise menu is included in the exercise program of the user (Step S2). In Step S2, the exercise performance determination unit 106 determines, for example, that, of the M types of exercise menus stored in the exercise menu information DB 103, the K types of exercise menus are included in the exercise program of the user. The exercise performance determination unit 106 notifies the exercise evaluation unit 108 of the exercise menus included in the exercise program of the user as the exercise menus to be evaluated.

The exercise information extraction unit 107 extracts exercise information depending on the type of the exercise data from exercise data of each user stored in the exercise record DB 102 (Step S3). For example, in Step S3, the exercise information extraction unit 107 extracts the exercise information from the exercise data regarding the exercise menus determined to be included in the exercise program in Step S2, or exercise menus to be evaluated in the exercise evaluation unit 108.

The exercise evaluation unit 108 determines whether or not there is an exercise menu that has not yet been evaluated in the exercise menus to be evaluated (Step S4). When there are exercise menus that have not yet been evaluated, the exercise evaluation unit 108 selects one of the exercise menus that have not yet been evaluated. The exercise evaluation unit 108 evaluates the selected exercise menu in accordance with the reference value. When evaluating the exercise menu, the exercise evaluation unit 108 calculates the rate of improvement from exercise information for each user who has performed exercise of each exercise menu to be evaluated in a predetermined period. The exercise evaluation unit 108 calculates the evaluation value of each exercise menu using the rate of improvement of each user. The exercise evaluation unit 108 sets, for example, the average of the evaluation values of all the exercise menus to be evaluated as the reference value.

The exercise evaluation unit 108 compares the evaluation value of the selected exercise menu with the reference value and determines whether the evaluation value of the selected exercise menu is equal to or larger than the reference value (Step S5). When it is determined that the evaluation value of the exercise menu selected in Step S5 is equal to or larger than the reference value, the process returns to Step S4, where the exercise evaluation unit 108 determines whether or not there is an exercise menu that has not yet been evaluated.

When it has been determined in Step S5 that the evaluation value is lower than the reference value, the exercise evaluation unit 108 adds the selected exercise menu to the list of low-quality exercise menus (Step S6). After that, the process returns to Step S4, where it is determined whether or not there is an exercise menu that has not yet been evaluated. When it has been determined in Step S4 that there is no exercise menu that has not yet been evaluated, the exercise evaluation unit 108 outputs the results of the evaluation (Step S7). In Step S7, the exercise evaluation unit 108 outputs the list of low-quality exercise menus that has been created as the results of the evaluation.

In this example embodiment, the exercise evaluation unit 108 evaluates exercise menus using the exercise information extracted from the exercise record data. In this example embodiment, the exercise menus can be evaluated by recording exercise performed on a daily basis. The exercise evaluation unit 108 outputs, for example, the results of the evaluation with which low-effective exercise menus can be identified. The manager is able to efficiently find low-effective exercise menus provided for the users from among a number of exercise menus stored in the exercise menu information DB 103 by referring to the results of the evaluation.

In this example embodiment, it is possible to evaluate exercise menus even when a user does not perform any special exercise such as a physical test. The manager is able to add or correct exercise menus based on the results of the evaluation using the terminal apparatus 110. According to this procedure, in the field of medicine or nursing care, each user is able to perform exercise menus having higher qualities. Further, the manager is able to obtain evaluation of exercise menus that reflect the state in which the user performs exercise on a daily basis even when the manager is not always present at the site of the exercise. Accordingly, the manager is able to provide exercise menus having higher qualities.

While the example in which the exercise record DB 102 stores data (sensor data) acquired by the sensor apparatus 101 as exercise data has been described in the above example embodiment, the present disclosure is not limited thereto. The exercise record DB 102 may store, for example, exercise information extracted from the sensor data that the sensor apparatus 101 acquires.

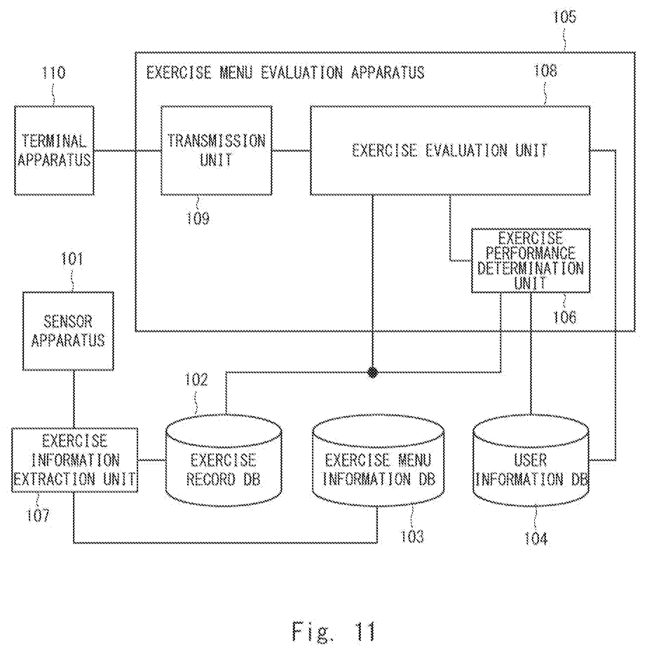

FIG. 11 shows a configuration example of an exercise menu evaluation apparatus according to a modified example. In this modified example, an exercise information extraction unit 107 extracts exercise information from the sensor data that the sensor apparatus 101 has acquired. The exercise information extraction unit 107 stores the extracted exercise information in the exercise record DB 102 as the exercise data. The exercise information extraction unit 107 may be a part of the exercise menu evaluation apparatus 105 or may be configured as an apparatus that is different from the exercise menu evaluation apparatus 105.

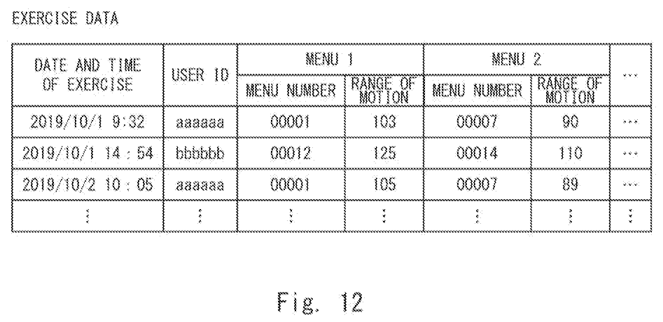

FIG. 12 shows data stored in the exercise record DB 102 in the above modified example. In this example, the exercise record DB 102 stores, for each of the exercise menus included in the exercise program, the menu number in association with the extracted exercise information (range of motion). In the above modified example, the exercise evaluation unit 108 is able to acquire, from the exercise record DB 102, the range of motion stored in association with the menu number of the exercise menu to be evaluated, thereby evaluating the exercise menus.

The exercise programs that include an exercise menu evaluated by the exercise menu evaluation apparatus 105 are not limited to exercise programs created by the exercise program providing apparatus 200. The exercise menu evaluation apparatus 105 may evaluate exercise menus included in exercise programs manually created by professionals such as medical therapists. Further, professionals may create exercise programs to be provided for users using the results of evaluating the exercise menus.

While the example in which the exercise evaluation unit 108 uses the average of the rates of improvement of each user as the reference value has been described in the above example embodiment, the present disclosure is not limited thereto. For example, the exercise evaluation unit 108 may use a value calculated by “average−standard deviation” as the reference value, assuming that the rate of improvement calculated for each exercise menu follows a normal distribution. In this case, the exercise evaluation unit 108 may determine that exercise menus whose evaluation values are smaller than the “average−standard deviation” are low-quality exercise menus. Alternatively, the reference value may be a value that the manager has input using the terminal apparatus 210.

The exercise evaluation unit 108 may classify the users or the exercise menus in accordance with desired attributes using the user information, the exercise menu information and the like and evaluate the exercise menus for each classification. The exercise evaluation unit 108 classifies users in accordance with, for example, the attributes such as sex, age, and medical history. Alternatively, the exercise evaluation unit classifies the K types of exercise menus in accordance with the attributes such as the target part. The exercise evaluation unit 108 calculates, for the K types of exercise menus, evaluation values for each classification. The exercise evaluation unit 108 determines, for each classification, a reference value based on the evaluation values calculated for each classification. For example, the exercise evaluation unit 108 sets the average of the evaluation values of the plurality of exercise menus calculated for one classification as the reference value in this classification. The exercise evaluation unit 108 may compare the evaluation values with the reference value for each classification and evaluate the exercise menus.

While the example in which the exercise evaluation unit 108 evaluates an exercise menu using exercise information of all the users who have performed the exercise menu to be evaluated has been described in the above example embodiment, the present disclosure is not limited thereto. The exercise evaluation unit 108 may evaluate the compatibility of a combination of exercise menus by taking the entire exercise program into consideration, not just independently evaluating one exercise menu.

For example, the exercise evaluation unit 108 calculates, using exercise information on the user for whom an exercise program including one exercise menu (a first exercise menu) and a specific exercise menu (a second exercise menu) are provided, the evaluation value of the first exercise menu. The exercise evaluation unit 108 compares the evaluation value of the first exercise menu with the reference value and generates the results of evaluating the first exercise menu. According to this procedure, it is possible to evaluate the effectiveness of the first exercise menu when the first exercise menu is combined with the second exercise program.

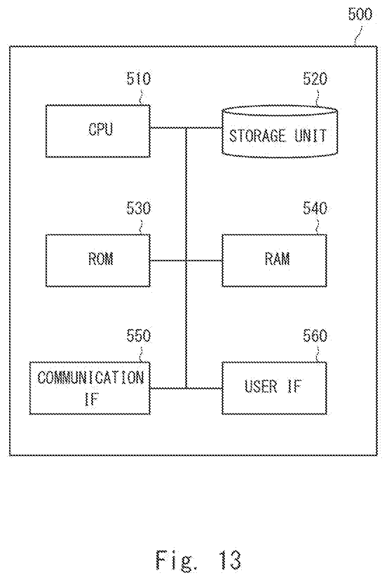

In the present disclosure, the exercise menu evaluation apparatus 105 and the exercise program providing apparatus 200 may be formed as a computer apparatus (server apparatus). FIG. 13 shows a configuration example of a computer apparatus that may be used as the exercise menu evaluation apparatus 105 or the exercise program providing apparatus 200. A computer apparatus 500 includes a control unit (CPU: Central Processing Unit) 510, a storage unit 520, a Read Only Memory (ROM) 530, a Random Access Memory (RAM) 540, a communication interface (IF: Interface) 550, and a user interface 560.

The communication interface 550 is an interface for connecting the computer apparatus 500 to a communication network via wired communication means, wireless communication means or the like. The user interface 560 includes a display unit such as a display. Further, the user interface 560 includes an input unit such as a keyboard, a mouse, and a touch panel.

The storage unit 520 is an auxiliary storage apparatus capable of holding various types of data. The storage unit 520 may not necessarily be a part of the computer apparatus 500, and may be an external storage apparatus, or a cloud storage connected to the computer apparatus 500 via a network.

The ROM 530 is a non-volatile storage apparatus. The ROM 530 may be, for example, a semiconductor storage apparatus such as a flash memory having a relatively small capacity. The program executed by the CPU 510 may be stored in the storage unit 520 or the ROM 530. The storage unit 520 or the ROM 530 stores various types of programs for implementing the functions of the respective parts in, for example, the exercise menu evaluation apparatus 105 or the exercise program providing apparatus 200.

The above program can be stored and provided to the computer apparatus 500 using any type of non-transitory computer readable media. Non-transitory computer readable media include any type of tangible storage media. Examples of non-transitory computer readable media include, for example, magnetic storage media such as flexible disks, magnetic tapes, hard disk drives, for example, optical magnetic storage media such as magneto-optical disks, optical disc media such as CD (compact disc), or DVD (digital versatile disk), and semiconductor memories such as mask ROM, programmable ROM (PROM), erasable PROM (EPROM), flash ROM, or RAM. Further, the program(s) may be provided to a computer using any type of transitory computer readable media. Examples of transitory computer readable media include electric signals, optical signals, and electromagnetic waves. Transitory computer readable media can provide the program to a computer via a wired communication line (e.g., electric wires, and optical fibers) or a wireless communication line.

The RAM 540 is a volatile storage apparatus. The RAM 540 may be a semiconductor memory device such as a Dynamic Random Access Memory (DRAM) or a Static Random Access Memory (SRAM). The RAM 540 may be used as an internal buffer that temporarily stores data or the like. The CPU 510 extends a program stored in the storage unit 520 or the ROM 530 to the RAM 540 and executes the program. The CPU 510 executes the program, whereby the functions of the respective parts in the exercise menu evaluation apparatus 105 or the exercise program providing apparatus 200 may be implemented. The CPU 510 may include an internal buffer capable of temporarily storing data and the like.

While the example embodiment of the present disclosure has been described in detail, the present disclosure is not limited to the aforementioned embodiment and a modification or a correction of the aforementioned embodiment without departing from the spirit of the present disclosure may also be included within the scope of the present disclosure.

The whole or part of the example embodiment disclosed above can be described as, but not limited to, the following supplementary notes.

### [Supplementary Note 1]

An exercise menu evaluation apparatus comprising:

evaluation value calculation means for calculating, based on time-series changes in exercise information on each of a plurality of users who have exercised in accordance with an exercise menu, an evaluation value of the exercise menu; and

evaluation result generation means for comparing the evaluation value of the exercise menu with a reference value and generating results of evaluating the exercise menu based on the results of the comparison.

### [Supplementary Note 2]

The exercise menu evaluation apparatus according to Supplementary Note 1, wherein the exercise information is information indicating a range of motion of a target part in the exercise menu.

### [Supplementary Note 3]

The exercise menu evaluation apparatus according to Supplementary Note 1 or 2, wherein the evaluation value calculation means calculates the evaluation value for each of a plurality of exercise menus, and the evaluation result generation means determines the reference value based on the evaluation values of the plurality of exercise menus.

### [Supplementary Note 4]

The exercise menu evaluation apparatus according to Supplementary Note 3, wherein the evaluation result generation means sets an average value of the evaluation values of the plurality of exercise menus as the reference value.

### [Supplementary Note 5]

The exercise menu evaluation apparatus according to Supplementary Note 3 or 4, wherein the evaluation result generation means classifies at least one of the plurality of users or the plurality of exercise menus according to attributes, calculates, for each classification, the evaluation values of the plurality of exercise menus, and determines the reference value for each classification based on the evaluation values calculated for each classification.

### [Supplementary Note 6]

The exercise menu evaluation apparatus according to any one of Supplementary Notes 1 to 5, wherein

the evaluation value calculation means calculates, based on time-series changes in exercise information on each of a plurality of users who have exercised in accordance with an exercise program including a first exercise menu and a second exercise menu, an evaluation value of the first exercise menu, and

the evaluation result generation means compares the evaluation value of the first exercise menu with a reference value and generates results of evaluating the first exercise menu based on the results of the comparison.

### [Supplementary Note 7]

The exercise menu evaluation apparatus according to any one of Supplementary Notes 1 to 6, wherein the evaluation result generation means determines that exercise menus whose evaluation values are smaller than the reference value are low-effective exercise menus.

### [Supplementary Note 8]

The exercise menu evaluation apparatus according to Supplementary Note 7, wherein the evaluation result generation means generates a list of low-effective exercise menus as the result of the evaluation.

### [Supplementary Note 9]

The exercise menu evaluation apparatus according to any one of Supplementary Notes 1 to 8, further comprising an exercise performance determination unit configured to determine whether or not the exercise menu is included in an exercise program of a user,

wherein the evaluation value calculation means calculates, for the exercise menu included in the exercise program, an evaluation value of the exercise menu.

### [Supplementary Note 10]

The exercise menu evaluation apparatus according to any one of Supplementary Notes 1 to 9, further comprising exercise information extraction means for extracting the exercise information from exercise data acquired using a sensor apparatus.

### [Supplementary Note 11]

The exercise menu evaluation apparatus according to Supplementary Note 10, wherein the sensor apparatus comprises a camera that captures images of a user who is exercising.

### [Supplementary Note 12]

The exercise menu evaluation apparatus according to any one of Supplementary Notes 1 to 11, wherein the evaluation value calculation means calculates the evaluation value based on the exercise information at a plurality of time points.

### [Supplementary Note 13]

The exercise menu evaluation apparatus according to Supplementary Note 12, wherein

the plurality of time points include a first time point, a second time point later than the first time point, and a third time point between the first time point and the second time point, and

the evaluation value calculation means calculates a rate of improvement indicating the effect of the exercise menu as the evaluation value based on an amount of change between exercise information at the first time point and exercise information at the second time point and an amount of change between exercise information at the first time point and exercise information at the third time point.

### [Supplementary Note 14]

The exercise menu evaluation apparatus according to Supplementary Note 13, wherein the first time point is a time point at which the user has first performed the exercise menu in a predetermined period, the second time point is a time point at which the predetermined period is ended, and the third time point is an intermediate time point between the first time point and the second time point.

### [Supplementary Note 15]

The exercise menu evaluation apparatus according to Supplementary Note 13 or 14, wherein the evaluation value calculation means provides, when there is no exercise information in at least one of the second time point or the third time point, a buffer period before and after at least one of the second time point or the third time point, and determines exercise information at at least one of the second time point or the third time point based on exercise information in the buffer period.

### [Supplementary Note 16]

The exercise menu evaluation apparatus according to Supplementary Note 15, wherein the evaluation value calculation means uses the best exercise information in the buffer period as exercise information at at least one of the second time point or the third time point.

### [Supplementary Note 17]

The exercise menu evaluation apparatus according to any one of Supplementary Notes 1 to 16, further comprising transmission means for transmitting the results of the evaluation to a terminal apparatus of a manager.

### [Supplementary Note 18]

An exercise menu evaluation method comprising:

calculating, based on time-series changes in exercise information on each of a plurality of users who have exercised in accordance with an exercise menu, an evaluation value of the exercise menu; and

comparing the evaluation value of the exercise menu with a reference value and generating results of evaluating the exercise menu based on the results of the comparison.

### [Supplementary Note 19]

A program for causing a computer to execute processing of:

calculating, based on time-series changes in exercise information on each of a plurality of users who have exercised in accordance with an exercise menu, an evaluation value of the exercise menu; and

comparing the evaluation value of the exercise menu with a reference value and generating results of evaluating the exercise menu based on the results of the comparison.

This application is based upon and claims the benefit of priority from Japanese Patent Application No. 2019-236956, filed on Dec. 26, 2019, the disclosure of which is incorporated herein in its entirety by reference.

### REFERENCE SIGNS LIST

10 Exercise Menu Evaluation Apparatus
11 Evaluation Value Calculation Means
12 Evaluation Result Generation Means
100 Exercise Program Presenting System
101 Sensor Apparatus
102 Exercise Record DB
103 Exercise Menu Information DB
104 User Information DB
105 Exercise Menu Evaluation Apparatus
106 Exercise Performance Determination Unit
107 Exercise Information Extraction Unit
108 Exercise Evaluation Unit
109 Transmission Unit
110 Terminal Apparatus
200 Exercise Program Providing Apparatus
210 Terminal Apparatus

What is claimed is:

1. An exercise menu evaluation apparatus comprising:
at least one memory storing instructions, and
at least one processor configured to execute the instructions to;
calculate, based on time-series changes in exercise information on each of a plurality of users who have exercised in accordance with an exercise menu, an evaluation value of the exercise menu; and
compare the evaluation value of the exercise menu with a reference value and generate results of evaluating the exercise menu based on the results of the comparison.

2. The exercise menu evaluation apparatus according to claim 1, wherein the exercise information is information indicating a range of motion of a target part in the exercise menu.

3. The exercise menu evaluation apparatus according to claim 1, wherein the at least one processor is further configured to execute the instructions to calculate the evaluation value for each of a plurality of exercise menus, and determine the reference value based on the evaluation values of the plurality of exercise menus.

4. The exercise menu evaluation apparatus according to claim 3, wherein the at least one processor is further configured to execute the instructions to set an average value of the evaluation values of the plurality of exercise menus as the reference value.

5. The exercise menu evaluation apparatus according to claim 3, wherein the at least one processor is further configured to execute the instructions to classify at least one of the plurality of users or the plurality of exercise menus according to attributes, calculate, for each classification, the evaluation values of the plurality of exercise menus, and determine the reference value for each classification based on the evaluation values calculated for each classification.

6. The exercise menu evaluation apparatus according to claim 1, wherein the at least one processor is further configured to execute the instructions to:
calculate, based on time-series changes in exercise information on each of a plurality of users who have exercised in accordance with an exercise program including a first exercise menu and a second exercise menu, an evaluation value of the first exercise menu, and
compare the evaluation value of the first exercise menu with a reference value and generates results of evaluating the first exercise menu based on the results of the comparison.

7. The exercise menu evaluation apparatus according to claim 1, wherein the at least one processor is further configured to execute the instructions to determine that exercise menus whose evaluation values are smaller than the reference value are low-effective exercise menus.

8. The exercise menu evaluation apparatus according to claim 7, wherein the at least one processor is further configured to execute the instructions to generate a list of low-effective exercise menus as the result of the evaluation.

9. The exercise menu evaluation apparatus according to claim 1, wherein the at least one processor is further configured to execute the instructions to:
determine whether or not the exercise menu is included in an exercise program of a user, and
calculate, for the exercise menu included in the exercise program, an evaluation value of the exercise menu.

10. The exercise menu evaluation apparatus according to claim 1, wherein the at least one processor is further configured to execute the instructions to extract the exercise information from exercise data acquired using a sensor apparatus.

11. The exercise menu evaluation apparatus according to claim 10, wherein the sensor apparatus comprises a camera that captures images of a user who is exercising.

12. The exercise menu evaluation apparatus according to claim 1, wherein the at least one processor is further configured to execute the instructions to calculate the evaluation value based on the exercise information at a plurality of time points.

13. The exercise menu evaluation apparatus according to claim 12, wherein
the plurality of time points include a first time point, a second time point later than the first time point, and a third time point between the first time point and the second time point, and
the at least one processor is further configured to execute the instructions to calculate a rate of improvement indicating the effect of the exercise menu as the evaluation value based on an amount of change between exercise information at the first time point and exercise information at the second time point and an amount of change between exercise information at the first time point and exercise information at the third time point.

14. The exercise menu evaluation apparatus according to claim 13, wherein the first time point is a time point at which the user has first performed the exercise menu in a predetermined period, the second time point is a time point at which the predetermined period is ended, and the third time point is an intermediate time point between the first time point and the second time point.

15. The exercise menu evaluation apparatus according to claim 13, wherein the at least one processor is further configured to execute the instructions to provide, when there is no exercise information in at least one of the second time point or the third time point, a buffer period before and after at least one of the second time point or the third time point, and determine exercise information at at least one of the second time point or the third time point based on exercise information in the buffer period.

16. The exercise menu evaluation apparatus according to claim 15, wherein the at least one processor is further configured to execute the instructions to use the best exercise information in the buffer period as exercise information at at least one of the second time point or the third time point.

17. The exercise menu evaluation apparatus according to claim 1, wherein the at least one processor is further configured to execute the instructions to transmit the results of the evaluation to a terminal apparatus of a manager.

18. An exercise menu evaluation method comprising:
calculating, based on time-series changes in exercise information on each of a plurality of users who have exercised in accordance with an exercise menu, an evaluation value of the exercise menu; and
comparing the evaluation value of the exercise menu with a reference value and generating results of evaluating the exercise menu based on the results of the comparison.

19. A non-transitory computer readable medium storing a program for causing a computer to execute processing of:
calculating, based on time-series changes in exercise information on each of a plurality of users who have exercised in accordance with an exercise menu, an evaluation value of the exercise menu; and
comparing the evaluation value of the exercise menu with a reference value and generating results of evaluating the exercise menu based on the results of the comparison.
